(19) United States
(12) Patent Application Publication
(10) Pub. No.: US 2022/0117895 A1
(43) Pub. Date: Apr. 21, 2022

(54) PREPARING LIPOSOMES WITH HIGH DRUG LOADING CAPACITY AND THE USE THEREOF

(71) Applicant: Purdue Research Foundation, West Lafayette, IN (US)

(72) Inventors: Yoon Yeo, West Lafayette, IN (US); Hassan Tamam, Sohag (EG)

(73) Assignee: Purdue Research Foundation, West Lafayette, IN (US)

(21) Appl. No.: 17/423,492

(22) PCT Filed: Jan. 16, 2020
(86) PCT No.: PCT/US20/13777

§ 371 Date: Jul. 16, 2021

Related U.S. Application Data
(60) Provisional application No. 62/793,270, filed on Jan. 16, 2019.

(51) Int. Cl. A61K 9/127; A61K 31/7068; A61K 31/704; A61K 31/282; A61K 33/243; A61K 31/337; A61K 31/4745; A61K 31/519; A61K 31/475

(52) U.S. Cl. CPC A61K 9/1278; A61K 9/1272; A61K 31/7068; A61K 31/704; A61K 31/475; A61K 33/243; A61K 31/337; A61K 31/4745; A61K 31/519; A61K 31/282

(57) ABSTRACT

The present invention generally relates to a method for preparing a drug-loaded liposome, in particular to a method for preparing a drug-loaded liposome manufactured using hypertonic loading or a combination of hypertonic loading and remote loading of one or more drugs. The invention described herein also pertains to pharmaceutical compositions and methods for treating cancers. Both the manufacturing processes and the pharmaceutical products are within the scope of this disclosure.

### CROSS-REFERENCE TO RELATED APPLICATIONS

The present U.S. patent application relates to and claims the priority benefit of U.S. Provisional Patent Application Ser. No. 62/793,270, filed Jan. 16, 2019, the content of which is hereby incorporated by reference in its entirety.

### GOVERNMENT SUPPORT CLAUSE

This invention was made with government support under EB017791 and CA232419, awarded by the National Institute of Health. The government has certain rights in the invention.

### TECHNICAL FIELD

The present invention generally relates to a method for preparing a drug-loaded liposome, in particular to a manufacturing process for preparing a drug-loaded liposome manufactured using hypertonic loading or a combination of hypertonic loading and remote loading of a drug.

### BACKGROUND

This section introduces aspects that may help facilitate a better understanding of the disclosure. Accordingly, these statements are to be read in this light and are not to be understood as admissions about what is or is not prior art.

Liposomal drug carriers are used to reduce non-specific side effects of systemic chemotherapy. An important feature of liposomes is the versatility: liposomes can carry both hydrophilic and lipophilic drugs, with the former in the aqueous core compartment and the latter in the lipid bilayer membrane, respectively. Hydrophilic drugs can be passively loaded in the aqueous core during the hydration of the lipid film as part of the hydrating medium. Alternatively, weak acid or base drugs can be incorporated into the core compartment of preformed liposomes via chemical gradients across the lipid bilayer, which induce the influx of a drug and the formation of an ion complex of the drug. This method, called ‘remote loading’, has been employed in liposomal formulations of doxorubicin (Dox), bringing significant improvement in pharmacokinetics and safety profiles of the drug in humans (Gubernator, J., Expert Opin. Drug Deliv. 2011, 8, 565-80; Barenholz, Y., J Control Release 2012, 160, 117-34).

While the remote loading method can encapsulate drugs with a higher loading efficiency (drug to liposome weight ratio) than the passive loading, it does not work for all the weak acid or base drugs. For example, gemcitabine (Gem), which can also form an ionic complex with sulfate, is not encapsulated in liposomes as efficiently as Dox by the remote loading method (Xu, H. et al., Pharm. Res. 2014, 31, 2583-92). Gem with a pKa value of pH 3.6 does not ionize in the acidic aqueous compartment of liposomes as extensively as Dox (pKa: pH 8.68); thus, the pH gradient across the membrane does not translate to a high concentration gradient of unionized Gem. Therefore, the maximum loading capacity of Gem that can be achieved by the remote loading is no higher than 1 wt %. The low drug loading efficiency is problematic for several reasons. First of all, a large loss of drug during the preparation is not economical. Secondly, inefficient drug loading necessitates the use of a large amount of polar lipids that may cause unintended biological effects (Yeo, Y. et al., AAPS J. 2015, 17, 1096-04). Moreover, the increased total dose due to the low drug loading increases the injection volume and/or the concentration of liposomes to the extent that they become the dose-limiting factors (Wilhelm, S. et al., Nat. Rev. Mater. 2016, 1, 16014; Ernsting, M. J., et al., J. Control Release, 2012, 162, 575-81). A large dose of liposomes has also met with an increased toxicity in mice, due to the delayed tissue distribution of liposomes accompanied by the extended circulation, which increases the chances of free drug leakage.

Consistent with the difficulty in efficient loading, no viable liposomal Gem product is currently available on the market. Nevertheless, there are several compelling reasons to develop liposomal Gem. Upon systemic administration, Gem undergoes rapid metabolism and renal clearance with a half-life of 8-17 min (Federico, C. et al., Int. J. Nanomedicine 2012, 7, 5423-36). Non-specific distribution of Gem induces serious side effects such as myelosuppression, neutropenia, thrombocytopenia, and anemia (Dasanu, C. A. Expert Opin. Drug Saf. 2008, 7, 703-16). Liposomal Gem has the potential to improve the bioavailability and safety of Gem. Moreover, Gem is broadly pursued as a combination therapy with Dox in the treatment of breast cancer and hepatocellular carcinoma for a synergistic effect (Rivera, E. et al., J. Clin. Oncol. 2003, 21, 3249-54; Vogus, D. R. et al., J. Control Release 2017, 267, 191-02; Anajafi, T., et al., Bioconjugate Chem. 2016, 27, 762-71; Liu, D., et al., Colloids Surf. B Biointerfaces 2014, 113, 158-68). Liposomal Gem will be a more effective counterpart of liposomal Dox in achieving co-localization of the drug combination. Therefore, there is a strong unmet need for efficient liposomal encapsulation of Gem.

### SUMMARY OF THE INVENTION

In some illustrative embodiments, the present invention relates to a method to manufacturing a drug-loaded liposome comprising the steps of:
a. preparing a solution of mixed lipids in an organic medium of chloroform and methanol;
b. evaporating said organic medium and forming a film of the mixed lipids of DPPC, cholesterol and DSPE-PEG2000;
c. hydrating said film of the mixed lipids with an aqueous medium;
d. sonicating and then extruding hydrated film of the mixed lipids through a plurality of membranes followed by centrifugation to afford liposome pellet; and
e. loading a drug by incubating said liposome pellet in a drug solution for a period of time at an elevated temperature to afford a drug-loaded liposome.

In some illustrative embodiments, the present invention relates to a method to prepare a drug-loaded liposome as disclosed herein, wherein said aqueous medium comprises sodium chloride at a concentration of about 400 to 600 mM.

In some illustrative embodiments, the present invention relates to a method to prepare a drug-loaded liposome as disclosed herein, wherein said aqueous medium comprises a phosphate-buffered saline.

In some illustrative embodiments, the present invention relates to a method to prepare a drug-loaded liposome as disclosed herein, wherein said aqueous medium comprises ammonium sulfate at a concentration of about 200 to 300 mM and sodium chloride at a concentration of about 400 to 600 mM.

In some illustrative embodiments, the present invention relates to a method to prepare a drug-loaded liposome as disclosed herein, wherein said drug solution comprises gemcitabine, doxorubicin, or a combination thereof.

In some illustrative embodiments, the present invention relates to a method to prepare a drug-loaded liposome as disclosed herein, wherein said drug solution comprises about 10 mg/mL of gemcitabine or doxorubicin.

In some illustrative embodiments, the present invention relates to a method to prepare a drug-loaded liposome as disclosed herein, wherein said drug solution comprises about 10 mg/mL of gemcitabine and about 10 mg/mL of doxorubicin.

In some illustrative embodiments, the present invention relates to a method to prepare a drug-loaded liposome as disclosed herein, wherein said drug solution comprises about 50 mg/mL of gemcitabine or doxorubicin in deionized water.

In some illustrative embodiments, the present invention relates to a method to prepare a drug-loaded liposome as disclosed herein, wherein said drug solution comprises a drug selected from the group consisting of gemcitabine; platinum compounds (carboplatin, cisplatin and oxaplatin), anthracyclines (doxorubicin and daunorubicin), paclitaxel, docetaxel, camptothecin derivatives, antimetabolites (methotrexate, cytarabine), and Vinca alkaloids (vincristine, vinblastine and vinorelbine).

In some illustrative embodiments, the present invention relates to a method to prepare a drug-loaded liposome as disclosed herein, wherein said drug solution comprises gemcitabine, platinum compounds (carboplatin, cisplatin and oxaplatin), anthracyclines (doxorubicin and daunorubicin), paclitaxel, docetaxel, camptothecin derivatives, antimetabolites (methotrexate, cytarabine), Vinca alkaloids (vincristine, vinblastine and vinorelbine), or a combination thereof.

In some illustrative embodiments, the present invention relates to a method to prepare a drug-loaded liposome as disclosed herein, wherein said drug solution comprises capecitabine, docetaxel, or a combination thereof.

In some illustrative embodiments, the present invention relates to a method to prepare a drug-loaded liposome as disclosed herein, wherein said drug solution comprises carboplatin, etoposide, or a combination thereof.

In some illustrative embodiments, the present invention relates to a method to prepare a drug-loaded liposome as disclosed herein, wherein said drug solution comprises cisplatin, fluorouracil, or a combination thereof.

In some illustrative embodiments, the present invention relates to a method to prepare a drug-loaded liposome as disclosed herein, wherein said drug solution comprises cisplatin, topotecan, or a combination thereof.

In some illustrative embodiments, the present invention relates to a method to prepare a drug-loaded liposome as disclosed herein, wherein said drug solution comprises vinorelbine, cisplatin, or a combination thereof.

In some illustrative embodiments, the present invention relates to a method to prepare a drug-loaded liposome as disclosed herein, wherein said drug solution comprises vinorelbine, carboplatin, or a combination thereof.

In some illustrative embodiments, the present invention relates to a product manufactured according to the methods disclosed herein.

In some illustrative embodiments, the present invention relates to a method for treating a patient with cancer comprising the step of administrating a therapeutically effective amount of a product manufactured according to the methods disclosed herein.

In some illustrative embodiments, the present invention relates to a pharmaceutical composition comprising the drug-loaded liposomes manufactured according to the methods disclosed herein, together with one or more diluents, excipients or carriers.

In some illustrative embodiments, the present invention relates to a pharmaceutical composition comprising the drug-loaded liposomes manufactured according to the methods disclosed herein, together with one or more diluents, excipients or carriers, wherein said pharmaceutical composition is for treating a patient with cancer.

In some other illustrative embodiments, the present invention relates to a drug-loaded liposome manufactured according to the steps of:
a. preparing a solution of mixed lipids in an organic medium of chloroform and methanol;
b. evaporating said organic medium and forming a film of the mixed lipids;
c. hydrating said film of the mixed lipids with an aqueous medium;
d. sonicating and then extruding hydrated film of the mixed lipids through a plurality of membranes followed by centrifugation to afford liposome pellet; and
e. incubating said liposome pellet in a drug solution for a period of time at an elevated temperature and affording a drug-loaded liposome.

In some other embodiments, the present invention relates to a drug-loaded liposome as disclosed herein, wherein said temperature is about 60° C.

In some other embodiments, the present invention relates to a drug-loaded liposome as disclosed herein, wherein said period of time ranges from about 6 to about 24 hours.

In some other embodiments, the present invention relates to a drug-loaded liposome manufactured according to the process as disclosed herein, wherein said mixed lipids comprise DPPC, cholesterol and DSPE-PEG2000 at a weight ratio of about 6:3:1.

In some other embodiments, the present invention relates to a drug-loaded liposome as disclosed herein, wherein said aqueous medium a phosphate-buffered saline.

In some other embodiments, the present invention relates to a drug-loaded liposome as disclosed herein, wherein said drug solution comprises a drug selected from the group consisting of Gemcitabine; platinum compounds (carboplatin, cisplatin and oxaplatin), anthracyclines (doxorubicin and daunorubicin), paclitaxel, docetaxel, camptothecin derivatives, antimetabolites (methotrexate, cytarabine), and Vinca alkaloids (vincristine, vinblastine and vinorelbine).

In some other embodiments, the present invention relates to a drug-loaded liposome as disclosed herein, wherein said drug solution comprises gemcitabine.

In some other embodiments, the present invention relates to a drug-loaded liposome as disclosed herein, wherein said drug solution comprises gemcitabine, platinum compounds (carboplatin, cisplatin and oxaplatin), anthracyclines (doxorubicin and daunorubicin), paclitaxel, docetaxel, camptothecin derivatives, antimetabolites (methotrexate, cytarabine), Vinca alkaloids (vincristine, vinblastine and vinorelbine), or a combination thereof.

In some other embodiments, the present invention relates to a drug-loaded liposome as disclosed herein, wherein said drug solution comprises gemcitabine, doxorubicin, or a combination thereof.

### BRIEF DESCRIPTION OF THE DRAWINGS

These and other features, aspects and advantages of the present invention will be better understood with reference to the following figures, descriptions and claims.

FIG. 1A shows overview of passive loading, remote loading, small volume loading, and hypertonic loading. FIGS. 1B-1D show envisioned mechanism of each method, Remote loading (FIG. 1B), Small Volume Loading (FIG. 1C); and Hypertonic Loading (FIG. 1D). d-N represents a weak base drug such as Gem.

FIGS. 2A-2B show TEM images of L_{RS}G with its blank L_{RS} (FIG. 2A) and L_{RH}G with its blank L_{RH} (FIG. 2B). Scale bar: 200 nm.

FIG. 3A shows in vitro release kinetics of L_{RS}G, L_{RH}G; and FIG. 3B shows in vitro release kinetics of L_{R}D at pH 5.5 and 7.4. n=3 independent and identical batches. Mean±standard deviation (s.d.).

FIG. 4A shows cytotoxicity of free Gem, L_{RS}G, and L_{RH}G. FIG. 4B shows cytotoxicity of free Dox and L_{R}D after 72 h incubation. n=3 identical and independent tests. Mean±s.d.

FIGS. 5A-5D show cytotoxicity of various conditions. FIG. 5A: free Gem, L_{RS}G, and L_{RH}G; FIG. 5B: free Dox and L_{R}D after short-term incubation. n=5 tests of a representative batch. Mean±s.d. Drug uptake by Huh7 after 3, 6, 12 and 24 h incubation with free Gem, L_{RS}G, and L_{RH}G (FIG. 5C), or free Dox and L_{R}D (FIG. 5D). n=3 (Gem) and 4 (Dox) independent and identical tests of a representative batch. Mean±s.d. *: p<0.05, **: p<0.01 by, ****: p<0.0001 by two-way ANOVA test followed by Sidak's multiple comparisons test.

FIGS. 6A-6B show confocal microscopic images of Huh7 cells incubated with free Dox (FIG. 6A) or 25-NBD cholesterol labeled (FIG. 6B) *L_{R}D for 10 min, 3 h or 10 h.+CQ: cells incubated with chloroquine (inhibitor of endosomal acidification) for 12 h prior to the addition of liposomal Dox. Scale bars: 50 μm.

FIGS. 7A-7B show cytotoxicity of various conditions. FIG. 7A, free Gem/Dox combinations on Huh7 cells given in different sequences and in different molar ratios (n=3 tests. Mean±s.d.), and FIG. 7B, free or liposomal Gem/Dox combinations on Huh7 cells given simultaneously in different molar ratios (n=3 tests. mean±s.d.).

FIG. 8A shows dosing schedule. FIG. 8B shows changes of individual Huh7 tumor volume. Blue: PBS (n=6); Red: Gem+Dox (n=8); Green: L_{RS}G+L_{R}D (n=7). FIG. 8C shows specific growth rate of Huh7 tumor: Δ log V/Δt (V: tumor volumes; t: time in days). **: p<0.01, ***: p<0.001 by Tukey's multiple comparisons test. FIG. 8D shows survival curve of the animals receiving PBS, Gem+Dox, or L_{RS}G+L_{R}D. ***: p<0.001, ****: p<0.0001 by Log-rank (Mantel-Cox) test.

### DETAILED DESCRIPTION

While the concepts of the present disclosure are illustrated and described in detail in the figures and the description herein, results in the figures and their description are to be considered as exemplary and not restrictive in character; it being understood that only the illustrative embodiments are shown and described and that all changes and modifications that come within the spirit of the disclosure are desired to be protected.

In some illustrative embodiments, the present invention relates to a method to prepare a drug-loaded liposome comprising the steps of:
a. preparing a solution of mixed lipids in an organic medium of chloroform and methanol;
b. evaporating said organic medium and forming a film of the mixed lipids;
c. hydrating said film of the mixed lipids with an aqueous medium;
d. sonicating and then extruding hydrated film of the mixed lipids through a plurality of membranes followed by centrifugation to afford liposome pellet; and
e. loading a drug by incubating said liposome pellet in a drug solution for a period of time at an elevated temperature to afford a drug-loaded liposome.

In some illustrative embodiments, the present invention relates to a method to prepare a drug-loaded liposome as disclosed herein, wherein said aqueous medium comprises sodium chloride at a concentration of about 400 to 600 mM.

In some illustrative embodiments, the present invention relates to a method to prepare a drug-loaded liposome as disclosed herein, wherein said aqueous medium comprises a phosphate-buffered saline.

In some illustrative embodiments, the present invention relates to a method to prepare a drug-loaded liposome as disclosed herein, wherein said mixed lipids comprise DPPC, cholesterol and DSPE-PEG2000.

In some illustrative embodiments, the present invention relates to a method to prepare a drug-loaded liposome as disclosed herein, wherein said mixed lipids comprise DPPC, cholesterol and DSPE-PEG2000 having a weight ratio of about 6:3:1.

In some illustrative embodiments, the present invention relates to a method to prepare a drug-loaded liposome as disclosed herein, wherein said aqueous medium comprises ammonium sulfate at a concentration of about 200 to 300 mM and sodium chloride at a concentration of about 400 to 600 mM.

In some illustrative embodiments, the present invention relates to a method to prepare a drug-loaded liposome as disclosed herein, wherein said drug solution comprises gemcitabine, doxorubicin, or a combination thereof.

In some illustrative embodiments, the present invention relates to a method to prepare a drug-loaded liposome as disclosed herein, wherein said drug solution comprises about 10 mg/mL of gemcitabine or doxorubicin.

In some illustrative embodiments, the present invention relates to a method to prepare a drug-loaded liposome as disclosed herein, wherein said drug solution comprises about 10 mg/mL of gemcitabine and about 10 mg/mL of doxorubicin.

In some illustrative embodiments, the present invention relates to a method to prepare a drug-loaded liposome as disclosed herein, wherein said drug solution comprises about 50 mg/mL of gemcitabine or doxorubicin in deionized water.

In some illustrative embodiments, the present invention relates to a method to prepare a drug-loaded liposome as disclosed herein, wherein said drug solution comprises a drug selected from the group consisting of gemcitabine; platinum compounds (carboplatin, cisplatin and oxaplatin), anthracyclines (doxorubicin and daunorubicin), paclitaxel, docetaxel, camptothecin derivatives, antimetabolites (methotrexate, cytarabine), and Vinca alkaloids (vincristine, vinblastine and vinorelbine).

In some illustrative embodiments, the present invention relates to a method to prepare a drug-loaded liposome as disclosed herein, wherein said drug solution comprises gemcitabine, platinum compounds (carboplatin, cisplatin and oxaplatin), anthracyclines (doxorubicin and daunorubicin), paclitaxel, docetaxel, camptothecin derivatives, antimetabolites (methotrexate, cytarabine), Vinca alkaloids (vincristine, vinblastine and vinorelbine), or a combination thereof.

In some illustrative embodiments, the present invention relates to a method to prepare a drug-loaded liposome as disclosed herein, wherein said drug solution comprises capecitabine, docetaxel, or a combination thereof.

In some illustrative embodiments, the present invention relates to a method to prepare a drug-loaded liposome as disclosed herein, wherein said drug solution comprises carboplatin, etoposide, or a combination thereof.

In some illustrative embodiments, the present invention relates to a method to prepare a drug-loaded liposome as disclosed herein, wherein said drug solution comprises cisplatin, fluorouracil, or a combination thereof.

In some illustrative embodiments, the present invention relates to a method to prepare a drug-loaded liposome as disclosed herein, wherein said drug solution comprises cisplatin, topotecan, or a combination thereof.

In some illustrative embodiments, the present invention relates to a method to prepare a drug-loaded liposome as disclosed herein, wherein said drug solution comprises docetaxel, carboplatin, or a combination thereof.

In some illustrative embodiments, the present invention relates to a method to prepare a drug-loaded liposome as disclosed herein, wherein said drug solution comprises docetaxel, cisplatin, or a combination thereof.

In some illustrative embodiments, the present invention relates to a method to prepare a drug-loaded liposome as disclosed herein, wherein said drug solution comprises ifosfamide, doxorubicin, or a combination thereof.

In some illustrative embodiments, the present invention relates to a method to prepare a drug-loaded liposome as disclosed herein, wherein said drug solution comprises etoposide, cisplatin, or a combination thereof.

In some illustrative embodiments, the present invention relates to a method to prepare a drug-loaded liposome as disclosed herein, wherein said drug solution comprises gemcitabine, capecitabine, or a combination thereof.

In some illustrative embodiments, the present invention relates to a method to prepare a drug-loaded liposome as disclosed herein, wherein said drug solution comprises gemcitabine, cisplatin, or a combination thereof.

In some illustrative embodiments, the present invention relates to a method to prepare a drug-loaded liposome as disclosed herein, wherein said drug solution comprises ifosfamide, carboplatin, etoposide, or a combination thereof.

In some illustrative embodiments, the present invention relates to a method to prepare a drug-loaded liposome as disclosed herein, wherein said drug solution comprises irinotecan, fluorouracil, folinic acid, or a combination thereof.

In some illustrative embodiments, the present invention relates to a method to prepare a drug-loaded liposome as disclosed herein, wherein said drug solution comprises oxaliplatin, fluorouracil, folinic acid, or a combination thereof.

In some illustrative embodiments, the present invention relates to a method to prepare a drug-loaded liposome as disclosed herein, wherein said drug solution comprises paclitaxel, carboplatin, or a combination thereof.

In some illustrative embodiments, the present invention relates to a method to prepare a drug-loaded liposome as disclosed herein, wherein said drug solution comprises pemetrexed, carboplatin, or a combination thereof.

In some illustrative embodiments, the present invention relates to a method to prepare a drug-loaded liposome as disclosed herein, wherein said drug solution comprises pemetrexed, cisplatin, or a combination thereof.

In some illustrative embodiments, the present invention relates to a method to prepare a drug-loaded liposome as disclosed herein, wherein said drug solution comprises docetaxel, cyclophosphamide, or a combination thereof.

In some illustrative embodiments, the present invention relates to a method to prepare a drug-loaded liposome as disclosed herein, wherein said drug solution comprises vinorelbine, cisplatin, or a combination thereof.

In some illustrative embodiments, the present invention relates to a method to prepare a drug-loaded liposome as disclosed herein, wherein said drug solution comprises vinorelbine, carboplatin, or a combination thereof.

In some illustrative embodiments, the present invention relates to a product manufactured according to the methods disclosed herein.

In some illustrative embodiments, the present invention relates to a method for treating a patient with cancer comprising the step of administrating a therapeutically effective amount of a product manufactured according to the methods disclosed herein.

In some illustrative embodiments, the present invention relates to a pharmaceutical composition comprising the drug-loaded liposomes manufactured according to the methods disclosed herein, together with one or more diluents, excipients or carriers.

In some illustrative embodiments, the present invention relates to a pharmaceutical composition comprising the drug-loaded liposomes manufactured according to the methods disclosed herein, together with one or more diluents, excipients or carriers, wherein said pharmaceutical composition is for treating a patient with cancer.

In some other illustrative embodiments, the present invention relates to a drug-loaded liposome manufactured according to the steps of:
a. preparing a solution of mixed lipids in an organic medium of chloroform and methanol;
b. evaporating said organic medium and forming a film of the mixed lipids comprising DPPC, cholesterol and DSPE-PEG2000 at a weight ratio of about 6:3:1;
c. hydrating said film of the mixed lipids with an aqueous medium;
d. sonicating and then extruding hydrated film of the mixed lipids through a plurality of membranes followed by centrifugation to afford liposome pellet; and
e. incubating said liposome pellet in a drug solution for a period of time at an elevated temperature and affording a drug-loaded liposome.

In some other embodiments, the present invention relates to a drug-loaded liposome as disclosed herein, wherein said aqueous medium comprises ammonium sulfate at a concentration of about 200 to 300 mM.

In some other embodiments, the present invention relates to a drug-loaded liposome as disclosed herein, wherein said temperature is about 60° C.

In some other embodiments, the present invention relates to a drug-loaded liposome as disclosed herein, wherein said period of time ranges from about 6 to about 24 hours.

In some other embodiments, the present invention relates to a drug-loaded liposome as disclosed herein, wherein said aqueous medium comprises sodium chloride at a concentration of about 400 to 600 mM.

In some other embodiments, the present invention relates to a drug-loaded liposome as disclosed herein, wherein said aqueous medium a phosphate-buffered saline.

In some other embodiments, the present invention relates to a drug-loaded liposome as disclosed herein, wherein said drug solution comprises a drug selected from the group consisting of Gemcitabine; platinum compounds (carboplatin, cisplatin and oxaplatin), anthracyclines (doxorubicin and daunorubicin), paclitaxel, docetaxel, camptothecin derivatives, antimetabolites (methotrexate, cytarabine), and Vinca alkaloids (vincristine, vinblastine and vinorelbine).

In some other embodiments, the present invention relates to a drug-loaded liposome as disclosed herein, wherein said drug solution comprises gemcitabine.

In some other embodiments, the present invention relates to a drug-loaded liposome as disclosed herein, wherein said drug solution comprises gemcitabine, platinum compounds (carboplatin, cisplatin and oxaplatin), anthracyclines (doxorubicin and daunorubicin), paclitaxel, docetaxel, camptothecin derivatives, antimetabolites (methotrexate, cytarabine), Vinca alkaloids (vincristine, vinblastine and vinorelbine), or a combination thereof.

In some other embodiments, the present invention relates to a drug-loaded liposome as disclosed herein, wherein said drug solution comprises gemcitabine, doxorubicin, or a combination thereof.

In some embodiments, the present invention relates to a pharmaceutical product manufactured according to the process disclosed herein, together with one or more diluents, excipients or carriers.

In some embodiments, the present invention relates to a pharmaceutical composition manufactured according to the process disclosed herein, together with one or more diluents, excipients or carriers.

In some embodiments, the present invention relates to a method for treating a patient of cancer comprising the step of administering to a patient in need of relief from said cancer a therapeutically effective amount of a pharmaceutical composition disclosed herein.

In some embodiments, the present invention relates to use of a pharmaceutical composition disclosed herein in the manufacture of a medicament for treating cancer in a subject.

In some other embodiments, the present invention relates to a pharmaceutical composition comprising nanoparticles of one or more compounds disclosed herein, together with one or more diluents, excipients or carriers.

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosages may be single or divided, and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in particular a human being.

An approach to improve liposomal encapsulation of Gem involves the incubation of preformed liposomes in a small volume of concentrated Gem solution, which keeps the external Gem at the saturation solubility and thus generates the maximum concentration gradient across the liposomal membrane. With the small volume loading method, the loading efficiency of Gem in the liposomes increased from 0.2 wt % to 4 wt % (Xu, H. et al., Pharm. Res. 2014, 31, 2583-92). Another approach uses lipophilic Gem prodrugs, such as valeroyl, heptanoyl, lauroyl and stearoyl linear acyl derivatives of Gem, for liposomal encapsulation, achieving a loading efficiency of up to 24 mol % (8.6 wt % as Gem).

In this study, we explore various strategies to further increase Gem loading in liposomes, including a new method, called hypertonic loading. This simple method utilizes high osmotic pressure across the lipid bilayer, which induces the influx of external water phase containing unionized Gem. The hypertonic loading and small volume loading methods are combined with remote loading to reduce the back diffusion of drug from liposomes. The optimized liposomal Gem is characterized with respect to the physicochemical properties and in vitro activities, as compared with liposomal Dox. The in vivo efficacy of liposomal Gem is tested in the context of combination therapy with liposomal Dox in a xenograft model of Huh7 hepatocellular carcinoma.

Results and Discussion

Effect of Preparation Methods on Liposomal Drug Loading

Gem-loaded liposomes by passive loading (LPG) showed a negligible drug loading of 0.14 wt %. However, variations in the drug loading method, such as remote loading, small volume loading, and hypertonic loading, increased the Gem loading efficiency to 3.7 wt % (L_{R}G), 3.8 wt % (L_{S}G), and 2.4 wt % (L_{H}G), respectively (Table 1). With the combination of remote loading and small volume or hypertonic loading (Table 2), the Gem loading efficiency further increased to 9.4±0.6 wt % (L_{RS}G) and 10.3±1.4 wt % (L_{RH}G). Dox-loaded liposomes, made by the remote loading method (L_{R}D), showed the loading efficiency of 21.3±2.5 wt %, which approaches the theoretical Dox content (5/(20+5)=20 wt %).

The remote loading relies on the pH gradient across the lipid bilayer created by the ionization of ammonium sulfate and the diffusion of ammonia, which provides a driving force for the influx of unionized drug. The internalized drug undergoes ionization in the acidic internal pH (3.6) and forms a stable sulfate complex, which is precipitated inside the liposomes. This principle works well for Dox³ but not for Gem. Due to the low pKa value (3.6), the extent of Gem ionization in the interior of liposomes (ionized/unionized=1) is not as high as Dox with a pKa value of 8.68 (ionized/unionized=120,000); therefore, the loading efficiency of L_{R}G (3.7 wt %) was much lower than that of L_{R}D (21.3 wt %). Of note, our loading efficiency of L_{R}G (3.7 wt %) is substantially higher than that reported by Xu et al (0.18 wt %) (Xu, H. et al., Pharm. Res. 2014, 31, 2583-92). This difference may be attributable to the fact that the Gem concentration in the exterior of the liposomes was kept 38 times higher than Xu's (10 mg/mL vs. 0.26 mg/mL), which helped increase the concentration gradient of unionized Gem across the lipid bilayer. This is consistent with the principle of small volume loading, which increased the Gem loading up to 3.8 wt % on the basis of the maximum concentration gradient. The hypertonic loading method also helped to enhance the loading efficiency of Gem. In this method, liposomes were filled with hypertonic sodium chloride solution (462 mM) and suspended in Gem solution (10 mg/mL: i.e., 38 mM). The difference in ionic strengths created a high osmotic pressure inside the liposomes, pulling Gem along with water into the aqueous core of the liposomes. The combination of remote loading and small volume loading or hypertonic loading further increased the Gem loading. This enhancement demonstrates that the benefit of remote loading can be maximized when combined with additional means to increase the influx of unionized Gem, be it through increasing the concentration gradient (via small volume loading) or the osmotic pressure gradient (via hypertonic loading).

The combination of remote loading and small volume loading was also used to co-encapsulate Dox and Gem (L_{RS}GD). The loading contents of Dox and Gem were 10.0±2.1 wt % and 4.2±1.0 wt %, respectively, about half the maximum drug contents of the liposomes loaded with Dox or Gem individually (L_{R}D; L_{RS}G or L_{RH}G) (Table 2). This reduction in drug loading efficiency may be explained by the competition between Gem and Dox for available ammonium sulfate.

Particle Characterization

The z-averages of liposomes measured by DLS were 210-220 nm (Table 2). The zeta potential measured in 1 mM phosphate buffer (pH 7.4) was consistently negative irrespective of the loaded drug, likely due to the presence of cholesterol that reduces the binding of sodium ions to the membrane surface. DSPE-PEG₂₀₀₀ may also have contributed to the negative charge. The particle size and zeta potential of Gem-loaded liposomes (L_{RS}G, L_{RH}G) did not change significantly over 3-month storage at 4° C. (Table 3). L_{R}D was also stable for the first 2 months but showed a slight increase in size in the third month. The drug content in the liposomes did not change during the storage indicating the stability of liposomal Gem (Table 3).

TABLE 1
Encapsulation efficiency of Gem-loaded liposomes

| | | Small | Hyper- | Drug loading | | | |
|---|---|---|---|---|---|---|---|
| | Remote | volume | tonic | efficiency | Z-average | Zeta potential | |
| Liposomes | loading | loading | loading | (wt %) | (d, nm) | (mV) | N* |
| L_{R}G | + | | | 3.7 | 205 | −30.8 | 1 |
| L_{S}G | | + | | 3.8 | 171 | −28.2 | 1 |
| L_{H}G | | | + | 2.4 | 184 | −29.2 | 1 |
| L_{RS}G | + | + | | 9.4 ± 0.6 | 217 ± 11 | −29.6 ± 4.0 | 3 |
| L_{RH}G | + | | + | 10.3 ± 1.4 | 219 ± 16 | −31.8 ± 2.6 | 3 |

*n: number of batches

TABLE 2
Physical properties of liposomes

| | | | | | Drug | | |
|---|---|---|---|---|---|---|---|
| | | | Small | | loading | | Zeta |
| | | Remote | volume | Hyper-tonic | efficiency | Z-average | potential |
| Liposomes | Drug | loading | loading | loading | (wt %)* | (d, nm) | (mV) |
| L_{RS}G | Gem | + | + | | 9.4 ± 0.6 | 217 ± 19 | −29.6 ± 4.0 |
| L_{RH}G | Gem | + | | + | 10.3 ± 1.4 | 219 ± 18 | −31.8 ± 2.6 |
| L_{R}G | Dox | + | | | 21.3 ± 2.5 | 215 ± 16 | −22.5 ± 3.3 |
| L_{RS}GD | Dox | + | + | | 10.1 ± 2.1 | 275 ± 1 | −28.7 ± 3.2 |
| | Gem | | | | 4.2 ± 1.0 | | |

*Drug loading efficiency: Mass of loaded drug/mass of liposomes. Data are presented as means ± standard deviations of 3 tests of a representative batch.

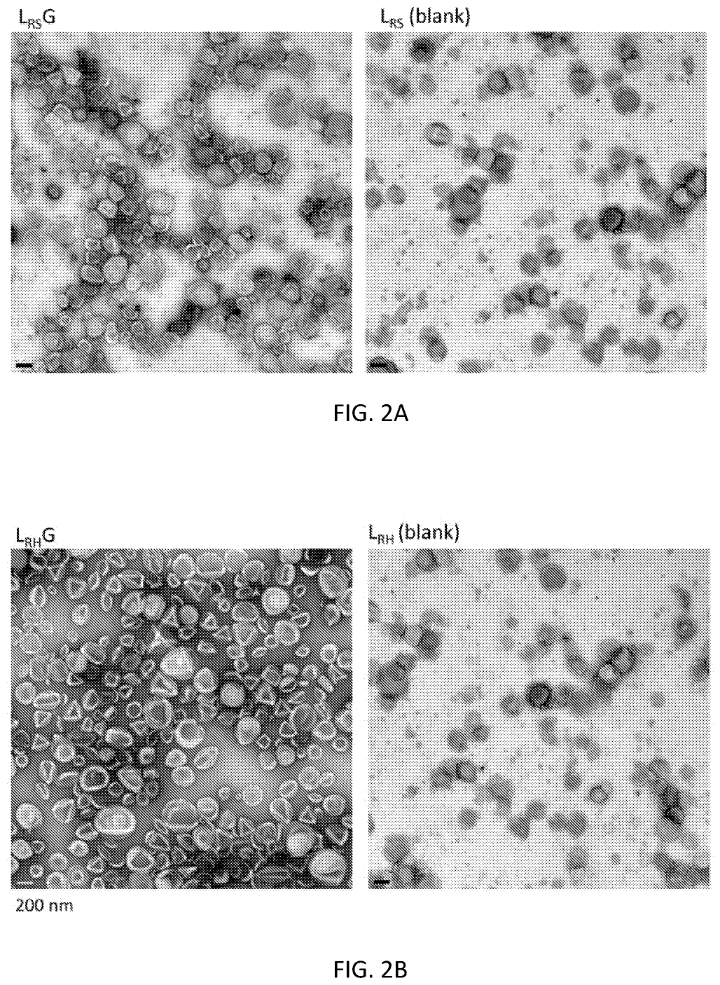

The particle size of the Gem-loaded liposomes (L_{RS}G, L_{RH}G) estimated by TEM using ImageJ was consistent with the DLS measurement, which ranged from 152 to 315 nm (FIGS. 2A-2B). In TEM micrographs, the interior of L_{RS}G and L_{RH}G appeared darker than blank counterparts, with many of them showing a triangular shape, which is likely Gem sulfate complex. L_{R}D liposomes were filled with rod-shape precipitates, typical of Dox sulfate complexes (Wei, X, et al., ACS Omega 2018, 3, 2508-17)

In Vitro Release Kinetics of Liposomal Drugs

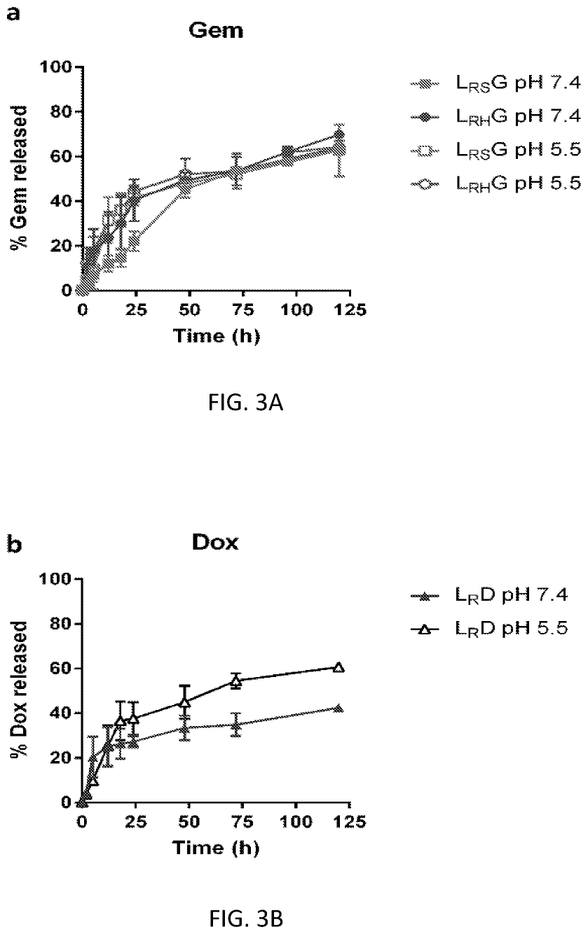

The drug release from liposomes were examined in phosphate-buffered saline (phosphate 10 mM) at pH 7.4 and pH 5.5, representing extracellular and the lysosomal pHs, respectively (FIGS. 3A-3B). Both L_{RS}G and L_{RH}G showed a sustained Gem release, irrespective of the pH (except for initial delay with L_{RS}G at pH 7.4), with ˜60% of the encapsulated Gem released by 120 h. The sustained release may be attributable to the formation of Gem sulfate complex, which does not readily pass the lipid bilayer. L_{R}D also showed a sustained release profile for a similar mechanism, but the extent of Dox release was pH-dependent (40% at pH 7.4 and 60% at pH 5.5 by 120 h). The differential Dox release may be explained by the relatively high solubility of Dox sulfate complex at acidic pH (Shibata, H. et al., Drug Dev. Ind. Pharm. 2015, 41, 1376-86).

TABLE 3
Physical stability of liposomes during storage

| | Storage | Initial | Size after | Initial zeta | Zeta | |
|---|---|---|---|---|---|---|
| | period | size | storage at | potential | potential | % Drug |
| Liposomes | (months) | (nm) | 4° C. (nm) | (mV) | (mv) | Content* |
| L_{RS}G | 1 | 218 ± 13 | 223 ± 2 | −31.8 ± 2.6 | −30.2 ± 1.8 | 98.1 ± 2.9 |
| | 2 | | 208 ± 6 | | −26.6 ± 6.0 | 99.0 ± 1.8 |
| | 3 | | 235 ± 1 | | −24.0 ± 1.6 | 98.8 ± 2.2 |
| L_{RH}G | 1 | 219 ± 19 | 220 ± 5 | −29.6 ± 4.0 | −32.1 ± 0.0 | 99.8 ± 3.0 |
| | 2 | | 207 ± 8 | | −32.2 ± 1.0 | 101.5 ± 2.0 |
| | 3 | | 197 ± 6 | | −25.5 ± 4.5 | 99.1 ± 1.0 |
| L_{R}D | 1 | 215 ± 16 | 210 ± 24 | −22.5 ± 3.3 | −22.0 ± 3.05 | 101.4 ± 1.2 |
| | 2 | | 237 ± 4 | | −17.5 ± 0.9 | 98.9 ± 2.0 |
| | 3 | | 259 ± 5 | | −14.5 ± 3.6 | 97.8 ± 1.0 |

*% Drug content = Drug content after storage/initial drug content × 100. Data are presented as the averages ± standard deviations of 3 independently and identically prepared batches

Cytotoxicity of Liposomal Drugs Vs. Free Drug Counterparts

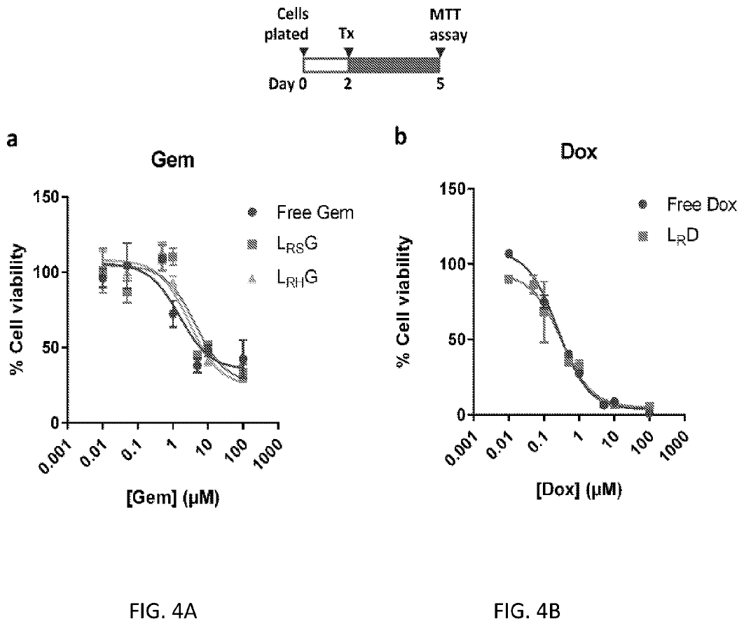

L_{RS}G, L_{RH}G and L_{R}D incubated with Huh7 cells for 72 h showed similar cytotoxicity as free drug counterparts (FIGS. 4A-4B) with comparable IC₅₀ values (1.6 μM, 4.3 μM, 3.0 μM for free Gem, L_{RS}G, and L_{RH}G; 0.25 μM and 0.32 μM for free Dox and L_{R}D). Blank liposomes did not show significant cytotoxicity. This result indicates that the liposomes released active drugs during the incubation.

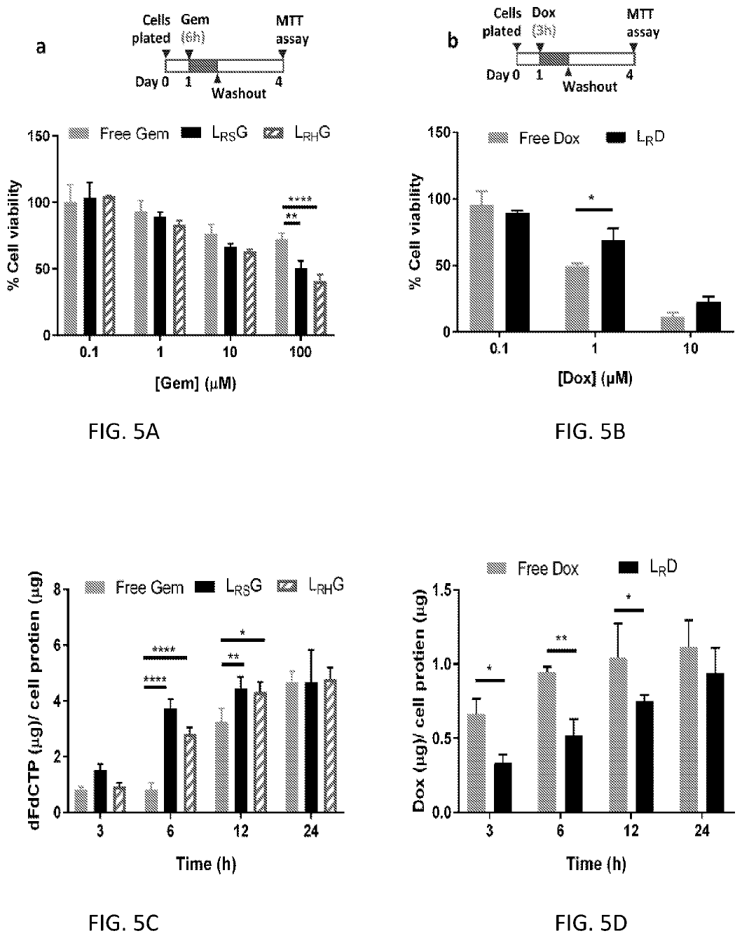

In another setting, the cells were exposed to the liposomes for a short time period (6 h for Gem; 3 h for Dox) to simulate dynamic in vivo environment, where the contact between the treatment and tumor cells declines with time. The cell viability after short-term exposure treatment reflects the effect of drug (as the released drug or liposomal drug) taken up by the cells during the exposure. With 6 h incubation, L_{RS}G and L_{RH}G showed greater toxicity than the equivalent dose of free Gem, reaching a statistical difference at 100 μM (FIG. 5A). Given that Gem release from the liposomes in the first 6 h was <20%, the greater toxicity of liposomal Gem may mainly be attributable to the improvement in cellular uptake of the drug. Free Gem is known to enter cells poorly due to the high hydrophilicity and the dependence on nucleoside transporters (Zakeri-Milani, P. et al., Excli. J. 2017, 16, 650-62; Ueno H. et al., Br. J. Cancer 2007, 97, 145). Liposomal Gem may be more efficient than free Gem as they can enter cells by diverse endocytic pathways (Kang, J. H. et al., Pharm. Res. 2017, 34, 704-17). In contrast, L_{R}D showed less toxicity than free Dox after 3 h exposure (significant difference shown at a concentration equivalent to Dox 1 μM, FIG. 5B), consistent with relatively slow Dox release. This suggests that liposomal Dox does not have advantage over free Dox as liposomal Gem does over free Gem in the cell level.

Cellular Uptake of Liposomal Drugs Vs. Free Drug Counterparts

To verify whether liposomal Gem enters cells more efficiently than free Gem (and liposomal Dox does the opposite), cellular uptake of each liposomes was investigated by measuring intracellular drug contents after timed incubation. As expected, Huh7 cells incubated with L_{RS}G or L_{RH}G showed greater intracellular concentration of dFdCTP (activated form of Gem: 2′,2′-difluoro-2′-deoxycytidine triphosphate) than those treated with free Gem (statistical difference shown at 6 and 12 h incubation, FIG. 5C). This result supports that the liposomal Gem improves the activity of drug by increasing the intracellular delivery of Gem. L_{R}D vs. free Dox showed the opposite trend, with free Dox entering cells more efficiently than L_{R}D, as evident at 3, 6, and 12 h (FIG. 5D). The attenuated cellular uptake coupled with slow drug release (FIG. 3B) may account for the relatively low activity of L_{R}D relative to free Dox after 3 h exposure (FIG. 5B).

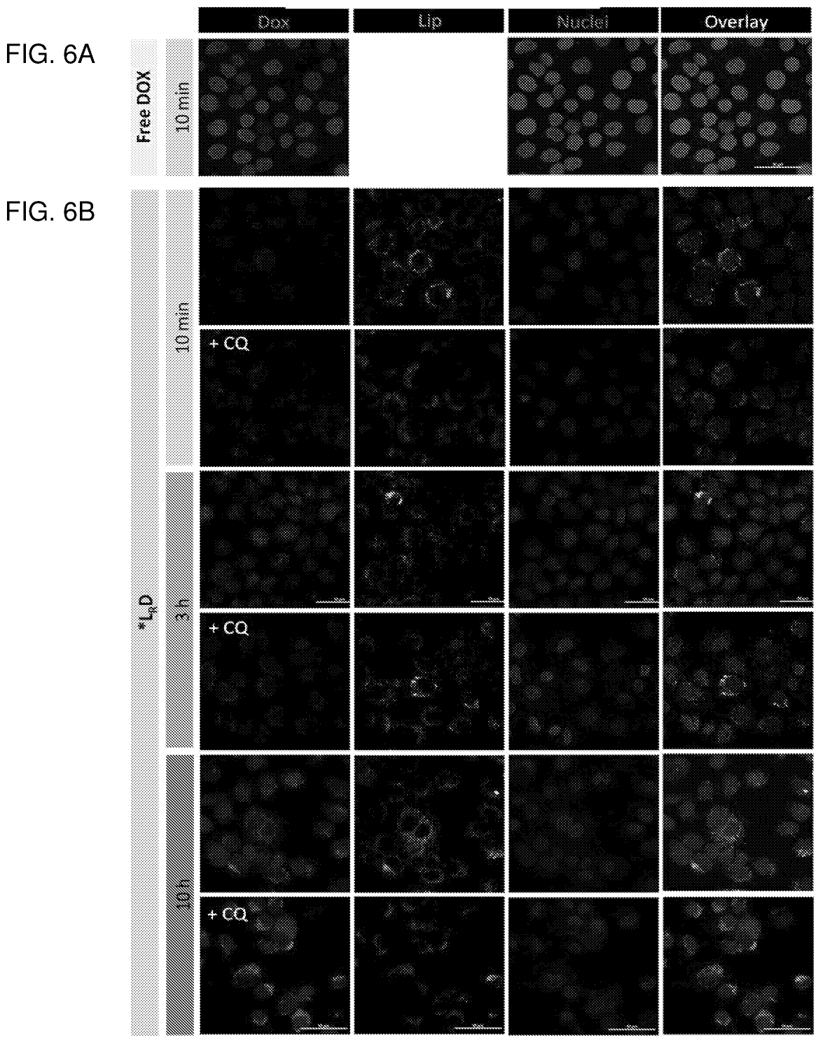

Confocal microscope imaging further confirmed the delayed cellular uptake and drug release of L_{R}D. Free Dox entered the cells more quickly than *L_{R}D (L_{R}D labeled with 25-NBD cholesterol), appearing in the nuclei as early as in 10 min (FIG. 6A). While the fluorescent cholesterol signal was observed in the cells in 10 min indicating the uptake of liposomes, Dox signal took longer to show up in the cells. When Dox was detected (at 3 h and 10 h), part of the signal was present in the cytosol in addition to the nucleus, reflecting the fraction of Dox remaining in the liposomes (FIG. 6B). When the cells were pretreated with chloroquine (CQ), which prevents the acidification of the lysosomes by consuming protons, Dox fluorescence appeared as punctate signals in the cytosol with little signal in the nuclei (Solomon, V. R. et al., Eur. J. Pharmacol. 2009, 625, 220-33). This indicates that *L_{R}D was taken up by endocytosis and trafficked to lysosomes and Dox release was delayed in the CQ-filled (hence less acidic) lysosomes, consistent with the acid-dependent release kinetics of liposomal Dox (FIGS. 3A-3B).

Optimization of Dox/Gem Combination

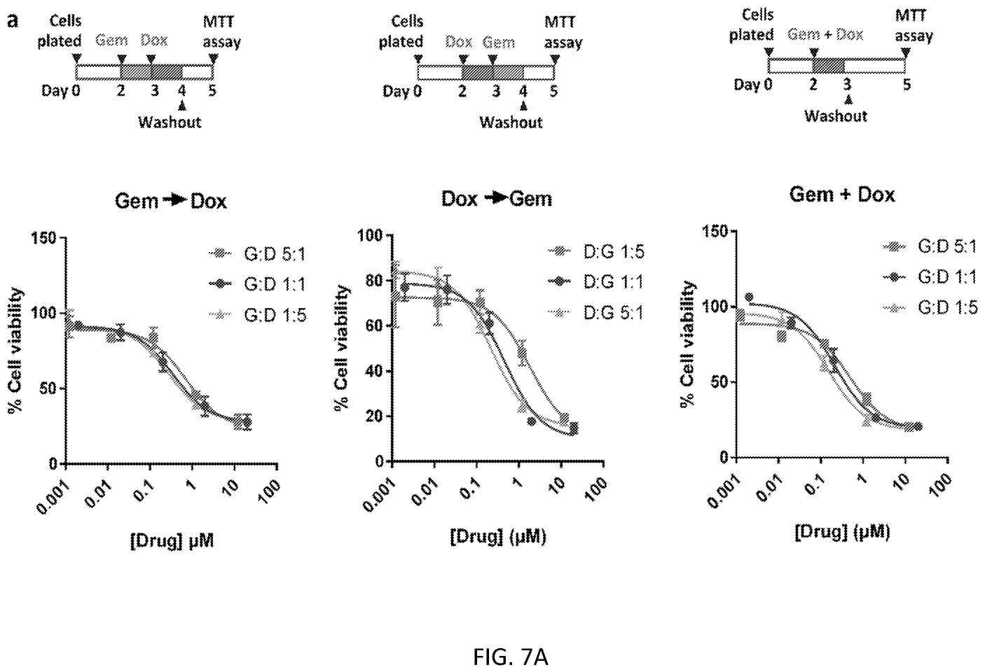

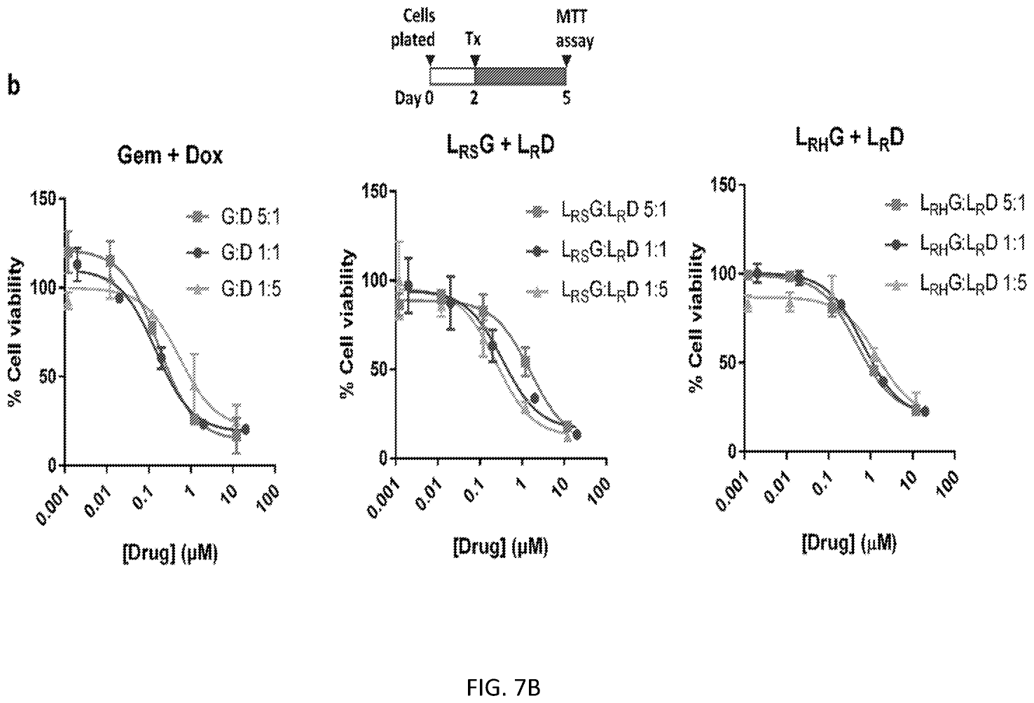

The bioactivity of liposomal Gem was tested in the context of combination therapy with Dox. To determine the optimal regimen for Gem/Dox combination treatment, free drug combinations were first tested with Huh7 cells in different sequences and ratios, keeping the exposure to each drug to 1 day. Simultaneous treatment (Gem+Dox) yielded relatively low CI values compared to sequential treatments at all ratios (FIG. 7A and Table 4). Accordingly, Huh7 cells were treated with combinations of liposomes simultaneously (L_{RS}G+L_{R}D or L_{RH}G+L_{R}D). The liposomal mixtures were found to be synergistic at all tested ratios (FIG. 7B and Table 5), when measured after 3 d exposure. L_{RS}G+L_{R}D combination at 1:1 molar ratio was selected for the following in vivo study.

TABLE 4
Combination indices of free drug combinations

| Molar ratio | Gem → Dox | Dox → Gem | Gem + Dox |
|---|---|---|---|
| (Gem:Dox) | (sequential) | (sequential) | (simultaneous) |
| 5:1 | 0.55 | 0.62 | 0.17 |
| 1:1 | 0.77 | 0.89 | 0.62 |
| 1:5 | 0.56 | 0.56 | 0.53 |

TABLE 5
Combination indices of liposomal drug combinations

| Molar ratio | | |
|---|---|---|
| (Gem:Dox) | L_{RS}G + L_{R}D | L_{RH}G + L_{R}D |
| 5:1 | 0.25 | 0.68 |
| 1:1 | 0.3 | 0.42 |
| 1:5 | 0.29 | 0.22 |

Anti-Tumor Efficacy of Liposomal Combination

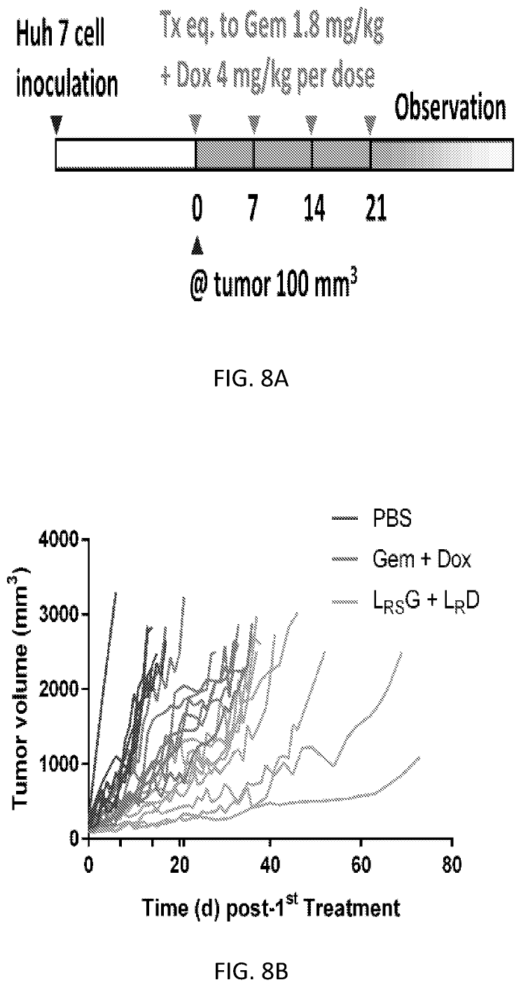

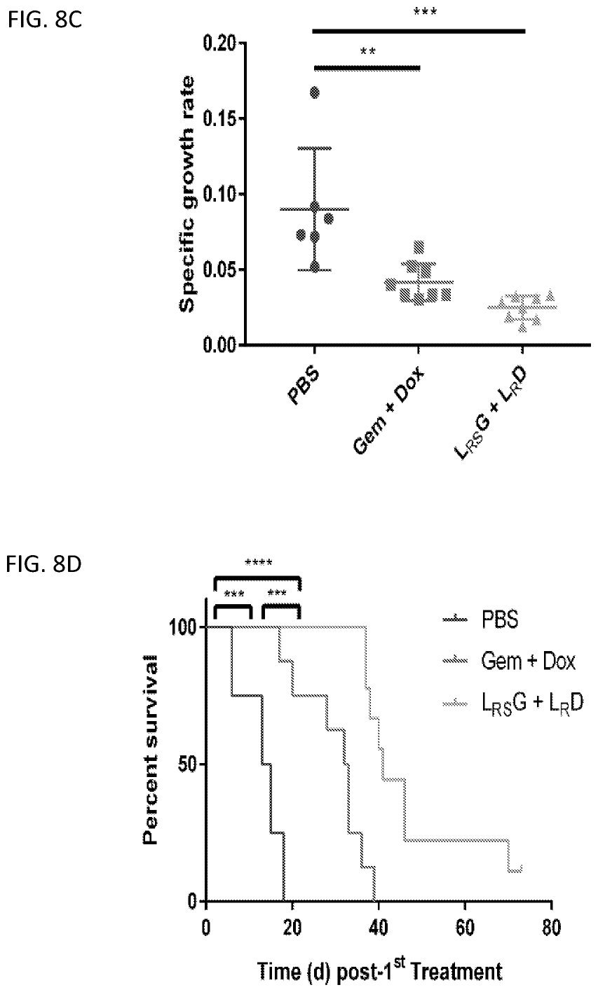

L_{RS}G+L_{R}D combination was simultaneously administered to male nude mice inoculated with subcutaneous Huh7 tumors at a q7d×4 schedule and compared with those treated with free drug combination at the equivalent dose (FIG. 8A). The total administered dose was below the reported maximum tolerated doses of Gem and Dox (16 mg/kg for Gem; 30 mg/kg for Dox) (Bornmann, C. et al., Cancer Chemother Pharmacol 2008, 61, 395-05). Both free drug and liposomal combinations were well tolerated without causing >20% weight loss during the treatment and induced significant delay in tumor growth as compared to PBS (free drug combination: p<0.01; liposomal combination: p<0.001 vs. PBS group, by Tukey's test) (FIG. 8C). The animals treated with the liposomal combination showed a significant extension in the median survival time compared with the PBS or free drug combination-treated animals (FIG. 8D; p<0.0001 vs. PBS group; p<0.001 vs. free drug combination group by Log-rank (Mantel-Cox) test). This improvement in anti-tumor effect is consistent with the enhanced tumor accumulation of liposomal drugs based on the enhanced permeability and retention effect (Ngoune, R. et al., J. Control Release 2016, 238, 58-70).

Conclusion: Liposomal Gem with high drug loading efficiency was produced by remote loading, small volume loading, hypertonic loading, and their combinations. Each method increased the loading efficiency from 0.14 wt % to 3.7 wt % (remote loading), 3.8 wt % (small volume loading), and 2.7 wt % (hypertonic loading), respectively. The combination of remote loading and small volume loading or hypertonic loading further increased the Gem loading efficiency to 9.4±0.6 wt % and 10.3±1.4 wt %, respectively, based on the increased influx and efficient entrapment of Gem in the liposomal core. The liposomal Gem showed high stability, sustained drug release, enhanced cellular uptake, and improved cytotoxicity as compared to free Gem. Liposomal Gem showed a synergistic effect with liposomal Dox on Huh7 hepatocellular carcinoma cells. A mixture of liposomal Gem and liposomal Dox attenuated tumor growth and extended the median survival time more efficiently than a free drug mixture relative to the control group in a xenograft model of Huh7 tumor. This study demonstrates the feasibility of producing bioactive liposomal Gem with an unprecedented high drug loading efficiency.

Materials and Methods

Dipalmitoyl-sn-glycero-3-phosphocholine (DPPC), cholesterol and N-(carbonyl-methoxypolyethylene-glycol-2000)-1,2-distearoyl-sn-glycero-3-phospho-ethanolamine (DSPE-PEG₂₀₀₀) were purchased from Avanti Polar Lipids (Alabaster, Ala.). Gemcitabine (Gem) and Doxorubicin HCl (Dox) were purchased from LC laboratories (Woburn, Mass.). Gemcitabine-5′-triphosphate (dFdCTP) was purchased from Sierra Bioresearch (Tucson, Ariz.). 3-(4,5-dimethylthiazol-2-yl)-2,5-diphenyltetrazolium bromide (MTT) was purchased from Invitrogen (Carlsbad, Calif.). All other materials, including solvents, were purchased from Sigma Aldrich (St. Louis, Mo.).

Liposome Preparation

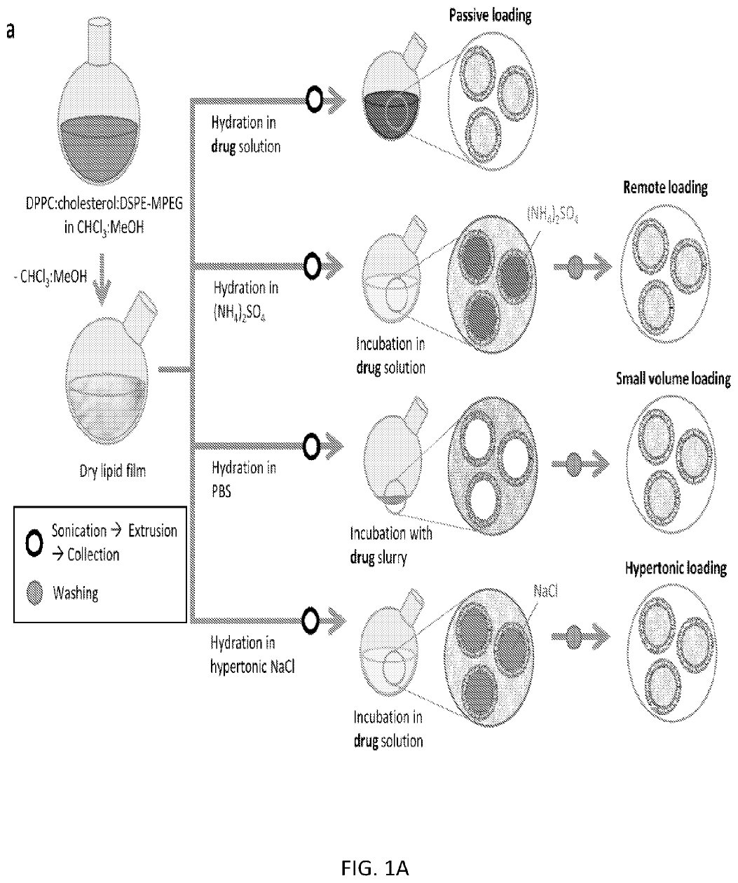

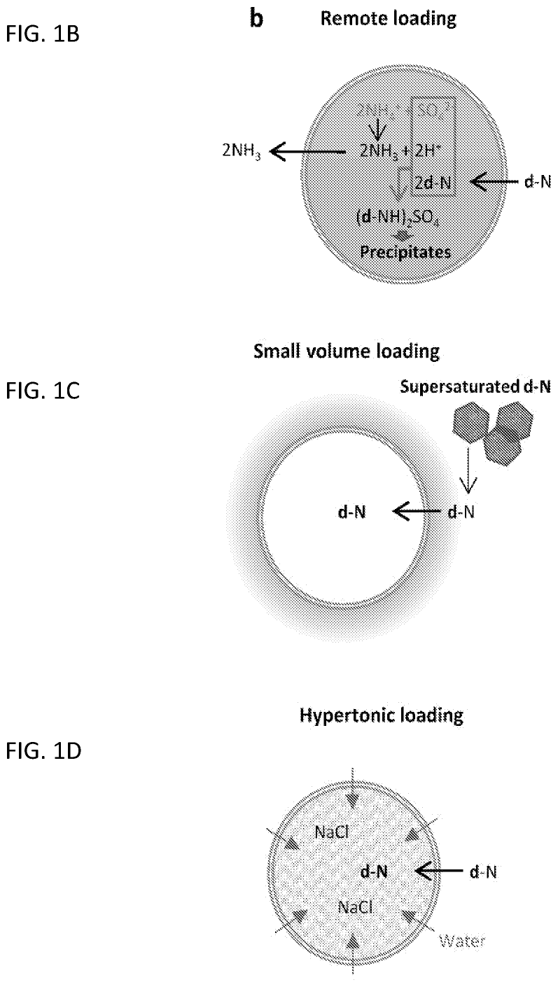

Liposomes were prepared in the following procedure with variations in hydration and drug loading methods (FIGS. 1A-1D). A mixture of DPPC, cholesterol, DSPE-PEG₂₀₀₀ at a weight ratio of 6:3:1 (20 mg in total) was dissolved in 3 mL of a 3:1 mixture of chloroform and methanol. For the preparation of fluorescently-labeled liposomes, 1 mg of cholesterol was replaced with 25-NBD cholesterol. A thin lipid film was obtained by removing the solvents with a rotary evaporator at 45° C. and hydrated according to the procedures detailed below. The hydrated lipid film was sonicated in a sonic water bath (Bransonic ultrasonic Co, Danbury, Conn.) for 15 min and extruded through polycarbonate membranes with a pore size of 400 nm and 200 nm, sequentially, using a Mini-extruder (Avanti polar lipid, Inc., AL). The drug-loaded liposomes were washed with deionized (DI) water 3 times by centrifugation at 135,700 rcf at 4° C. and used as is unless specified otherwise. In all methods, each batch used 20 mg of lipid components and 5 mg of Gem and/or 5 mg of Dox (theoretical loading efficiency of 20 wt %).

Passive loading: The lipid film was hydrated with 1 mL of 5 mg/mL Gem solution and stirred in a rotary evaporator for 45 min at 45° C., extruded and washed. The Gem-loaded liposomes prepared by this method are called LPG.

Remote loading: The lipid film was hydrated 1.2 mL of 250 mM ammonium sulfate solution and stirred in a rotary evaporator for 60 min at 45° C. The hydrated film was bath sonicated, extruded and collected by centrifugation at 305,400 rcf. The liposomal pellet was dispersed in 0.5 mL of 10 mg/mL Gem or Dox solution by bath sonication and incubated at 60° C. overnight. The Gem- or Dox-loaded liposomes prepared by remote loading are called L_{R}G and L_{R}D.

Small volume loading: The lipid film was hydrated in 1.2 mL of phosphate-buffered saline (PBS, pH 7.4). The hydrated film was bath sonicated for 10 min, extruded and collected by centrifugation at 305,400 rcf. The liposomal pellet was mixed with 0.1 mL of DI water containing 5 mg of Gem by 15 min bath sonication, followed by overnight incubation at 60° C. The Gem-loaded liposomes prepared by small volume loading are called L_{S}G.

Hypertonic loading: The lipid film was hydrated in 1.2 mL of 462 mM sodium chloride solution. The hydrated film was bath sonicated, extruded and collected by centrifugation at 305,400 rcf. The liposomal pellet was incubated in 0.5 mL of DI water containing 5 mg of Gem at 60° C. overnight. The Gem-loaded liposomes prepared by hypertonic loading are called L_{H}G.

Combination of Remote loading and Small volume loading: The liposomal pellet prepared by the remote loading method was mixed with 0.1 mL of DI water containing 5 mg of Gem by 15 min bath sonication, followed by overnight incubation at 60° C. The Gem-loaded liposomes prepared by the combination of remote loading and small volume loading are called L_{RS}G. Gem/Dox-coloaded liposomes (L_{RS}GD) were prepared by incubating the pellet in 0.1 mL of DI water containing 5 mg Gem and 5 mg Dox at 60° C. overnight.

Combination of Remote loading and Hypertonic loading: The lipid film was hydrated in 1.2 mL of DI water containing 250 mM Ammonium sulfate and 462 mM of sodium chloride. The liposomal pellet was incubated in 0.5 mL DI water containing 5 mg of Gem at 60° C. overnight. The Gem-loaded liposomes prepared by the combination of remote loading and hypertonic loading are called L_{RH}G.

Liposome Characterization

The z-average and zeta potential of each liposomal formulation were measured by a Malvern Zetasizer Nano ZS90 (Worcestershire, UK), as dispersed in DI water (z-average) or in 1 mM phosphate buffer (pH 7.4) (zeta potential). The liposomes were observed by the Tecnai F20 transmission electron microscope (FEI, Hillsboro, Oreg.) after negative staining with 1% uranyl acetate (Gem-loaded liposomes) or 1% phosphotungstic acid (Dox- or co-loaded liposomes). The size of particles in each micrograph was estimated by NIH ImageJ software (Bethesda, Md.).

Drug Loading Efficiency

The purified liposomes were lyophilized, accurately weighed, dispersed in 1 mL of acetonitrile and bath-sonicated in cold water for 2 h. The suspension was diluted with an equal volume of DI water and centrifuged at 16100 rcf for 20 min to obtain a clear supernatant. The supernatant was filtered through a 0.45 μm syringe filter and analyzed by high-pressure liquid chromatography (HPLC). HPLC analysis was performed by the Agilent 1100 system (Agilent Technologies, Palo Alto, Calif.), equipped with a C18 column (25 cm×4.6 mm, particle size 5 μm) (Supelco, St. Louis, Mo.). Dox was eluted with a 70:30 mixture of water and acetonitrile with 0.1% trifluoroacetic acid at a flow rate of 0.8 mL/min and detected at 369 nm. Gem was eluted with a 90:10 mixture of water and acetonitrile at a flow rate of 1 mL/min and detected at 269 nm (Liu, Y. et al., AAPS PharmSciTech 2018, 19, 207-14). The drug loading efficiency (%) is defined as W_{D}/W_{L}×100, where W_{D} is the amount of drug detected and W_{L} the total amount of the liposomes analyzed.

In Vitro Release Kinetics of Drug-Loaded Liposomes

Liposomes equivalent to 115 μg/mL of Gem or 250 μg/mL of Dox were placed in a Float-A-Lyzer G2 dialysis device (Spectrum Laboratories, Inc., Rancho Dominguez, Calif.) with a molecular weight cut-off of 100 kDa. The device was incubated in 20 mL of PBS (pH 7.4 or pH 5.5) at 37° C. with constant agitation. At predetermined time points, 0.3 mL of the release medium was sampled and replaced with 0.3 mL of fresh buffer. The sampled buffer was filtered with a syringe filter (0.45 μm pore size) and analyzed by HPLC.

Cytotoxicity of Drug-Loaded Liposomes

Huh7 human hepatocellular carcinoma cells (donation of Prof. Wanqing Liu) were cultured in RPMI-1640 medium complemented with 10% FBS, 100 U/mL penicillin, and 100m/mL streptomycin at 37° C. in a humidified 5% CO₂ atmosphere. Huh7 cells were seeded in a 96 well plate at a density of 10,000 cells per well and grown to 70% confluence. Huh7 cells were exposed to free drug or liposomal preparations for 3 or 6 h. The cells were rinsed twice with fresh medium and kept in drug-free medium for 72 h. In another experiment, Huh7 cells were incubated with a free Gem, L_{RS}G, L_{RH}G, free Dox, L_{R}D, blank liposomes, free drug mixture, or liposome mixture for 72 h. The medium was then removed, and 15 μL of 5 mg/mL MTT solution and 100 μL media were added to each well and incubated for 4 h at 37° C. Each well was then treated with 100 μL of stop/solubilization solution and agitated at 37° C. overnight. The absorbance of dissolved formazan was measured at 562 nm by a SpectraMax M3 microplate reader (Molecular Devices, Sunnyvale, Calif.).

Determination of Optimal Sequence of Combination Treatments

For determination of the optimal sequence of drug treatments, the cells were incubated with free Gem on the first day and free Dox on the second day (or vice versa), with the third day in drug-free medium (Gem→Dox or Dox→Gem). Alternatively, the cells were incubated with a Gem/Dox on the first day followed by 2 d incubation in drug-free medium (Gem+Dox). MTT assay was performed after the three days. The half maximal inhibitory concentration (IC₅₀) of each treatment was determined by GraphPad prism 7 (San Diego, Calif.). The combination index (CI) of each treatment was determined by Compusyn (Combosyn, Inc., Paramus, N.J.). The values of CI<1, CI=1, and CI>1 represent synergy, additivity, and antagonism, respectively.

Cellular Uptake of Drug-Loaded Liposomes

Quantitative Analysis

Huh7 Cells were seeded in a 12 well plate at a density of 10⁵ cells per well. After overnight incubation, the cells were treated with L_{RH}G, L_{RS}G, or free Gem at a concentration equivalent to 50 μM Gem, or with L_{R}D or free Dox at a concentration equivalent to 50 μM Dox. At 3, 6, 12, and 24 h post-treatment, the cells were rinsed twice with cold PBS, trypsinized and collected by centrifugation at 233 rcf. The cell pellets were suspended in PBS and lysed by three cycles of freezing and thawing followed by probe sonication. The protein content in each cell lysate was measured by micro BCA assay. A hundred microliters of cell lysate was mixed with 200 μL of acetonitrile, bath sonicated for 1 h, and centrifuged at 16100 rcf for 30 min to separate a supernatant. The supernatant was evaporated under vacuum overnight and reconstituted in 100 μL of PBS. Dox was detected by the microplate reader at λ_{Ex}/λ_{Em} of 488 nm/580 nm. dFdCTP was detected by HPLC using a 64:36 v/v mixture of two aqueous mobile phases: (i) KH₂PO₄ 10 mM, tetra butyl ammonium bromide (TBABr) 10 mM, pH 7 and 0.25% methanol and (ii) KH₂PO₄ 250 mM, TBABr 10 mM, pH 7 and 15% methanol, run at 1.2 mL/min, and a detection wavelength of 271 nm.

Confocal Imaging

Huh7 were seeded in a 35 mm glass-bottomed dish (Mat Tek Corp., Ashland, Mass.) at a density of 100,000 cells per dish. At 70% confluence, the cells were treated with free Dox for 10 min or fluorescently labeled liposomes (*L_{R}D, labeled with 25-NBD cholesterol) for 10 min, 3 h or 10 h. At each time point, the cells were rinsed twice with PBS. After nuclei staining with Hoechst 33342 (5m/mL) for 10 min, the cells were rinsed again with PBS and imaged in medium by a Nikon-A1R confocal microscope (Nikon America Inc., Melville, N.Y.). For selected treatments, chloroquine was added 12 h prior to the treatment. The cells were rinsed with PBS and incubated with the labeled liposomes for confocal imaging.

Anti-Tumor Efficacy of Liposomal Combination

All the animal procedures were approved by Purdue Animal Care and Use Committee, in conformity with the NIH guideline for the care and use of laboratory animals. 4-5 week old male athymic nude mice (Foxn1nu) were purchased from Envigo (Indianapolis, Ind.) and acclimatized for 4 days prior to the procedure. Each mouse received a subcutaneous injection of 10⁷ Huh7 cells in the upper flank of the right hind leg. The tumor length (L) and width (W) were measured by a digital caliber, and tumor volume (V) was calculated according to the equation: V=(L×W²)/2. When the tumor volume reached 100 mm³ (˜21 days after inoculation), the mice were randomized to 3 groups and treated with PBS (n=6), a free drug mixture comprising 4 mg/kg/dose of Dox and 1.8 mg/kg/dox Gem (n=8), or a mixture of their liposomal counterparts at the same doses (n=8) by tail-vein injection. Each treatment was repeated four times with a 7 day interval (q7d×4). The tumor volume and body weight were monitored daily. The tumor specific growth rate was calculated as Δ log V/Δt (t: time in days). Animals with tumors reaching more than 10% of the body weight were humanely euthanized.

Separate Description of Hypertonic Loading

Blank liposomes are filled with hypertonic sodium chloride solution (>300 mM) and suspended in drug solution (for example, 10 mg/mL gemcitabine solution). The difference in ionic strengths creates a high osmotic pressure inside the liposomes, pulling gemcitabine along with water into the aqueous core of the liposomes.

Hypertonic loading procedure: A mixture of DPPC, cholesterol, DSPE-PEG₂₀₀₀ at a weight ratio of 6:3:1 (20 mg in total) is dissolved in 3 mL of a 3:1 mixture of chloroform and methanol. A thin lipid film is obtained by removing the solvents with a rotary evaporator at 45° C. and hydrated in 1.2 mL of 462 mM sodium chloride solution. The hydrated lipid film is sonicated in a sonic water bath (Bransonic ultrasonic Co, Danbury, Conn.) for 15 min, extruded through polycarbonate membranes with a pore size of 400 nm and 200 nm, sequentially, using a Mini-extruder (Avanti polar lipid, Inc., AL), and collected by centrifugation at 305,400 rcf. The liposomal pellet is incubated in 0.5 mL of DI water containing 5 mg of gemcitabine at 60° C. overnight. The drug-loaded liposomes are washed with deionized (DI) water 3 times by centrifugation at 135,700 rcf at 4° C.

Combination of Remote loading and Hypertonic loading: The combination of remote loading and small volume loading or hypertonic loading further increases the drug loading. The lipid film is prepared as above and hydrated in 1.2 mL of DI water containing 250 mM Ammonium sulfate and 462 mM of sodium chloride. The liposomal pellet is incubated in 0.5 mL DI water containing 5 mg of Gem at 60° C. overnight. The drug-loaded liposomes are washed with DI water 3 times by centrifugation at 135,700 rcf at 4° C.

To those persons skilled in the art, the candidate drugs that can be reasonably encapsulated into the liposomes include gemcitabine; platinum compounds (cisplatin and oxaplatin), anthracyclines (doxorubicin and daunorubicin), paclitaxel, docetaxel, camptothecin derivatives, antimetabolites (methotrexate, cytarabine), and Vinca alkaloids (vincristine, vinblastine and vinorelbine)

To those persons skilled in the art, the methods of coencapsulation as disclosed herein can be carried out following the cited references. In chemotherapy of cancer, it is common to use combinations of drugs (Meng, F., Han, N. & Yeo, Y. Expert opinion on drug delivery 2017, 14, 427-446; Miao, L., et al. Adv Drug Deliv Rev 2017, 115, 3-22; Hu, Q., Sun, W., Wang, C. & Gu, Z. Adv Drug Deliv Rev 2016, 98, 19-34). The rationale of combination therapy is multifaceted. First, cancer cells carry multiple abnormalities, which may not be addressed by a single drug. Thus, cytotoxic drugs with different molecular targets are often combined to avoid the development of drug resistance. For example, paclitaxel and gemcitabine, which are inhibitors of microtubule depolymerization and DNA synthesis, respectively, are used together as the first-line treatment of metastatic pancreatic cancer (Saif, M W, JOP 2013, 14(6): 686-688). In addition, cancer cells treated with a single drug can use alternative survival pathways and become resistant to the treatment (Hanahan, D, et al., Cell 2011, 144, 646-674). Therefore, experimental studies have used combinations of chemotherapy and gene therapeutics targeting various aspects of tumor progression (Teo, P Y, et al., Adv. Drug Deliv Rev 2016, 98, 41-63). For example, siRNA targeting P-glycoprotein has been used in combination with paclitaxel and doxorubicin to overcome drug resistance of tumor cells (Xiao, B. et al, Expert Opinion Drug Delivery 2017, 14, 65-73; Huang, W, et al., Adv. Drug Deliv Rev 2017, 115, 82-97). Emerging new therapeutics can also benefit from combination therapy. Co-administration of a transforming growth factor-β inhibitor facilitates cytotoxic T-cell infiltration into tumors and significantly enhances immune checkpoint blockade therapy of mammary carcinoma (Mariathasan, S. et al., Nature 2018, 554, 544). A recent study also finds that the microenvironment limits therapeutic effects of gemcitabine by harboring bacteria that produce drug-inactivating enzymes (Geller, L T, et al., Science 2017, 357, 1156-1160). In this case, co-administration of antibiotics is warranted to enhance gemcitabine-based chemotherapy.

To those persons skilled in the art, a combination of two or more drug products into the liposomes manufactured according to the methods disclosed herein, those combination drug candidates may comprise: Gemcitabine+doxorubicin; Capecitabine+docetaxel; Carboplatin+etoposide; Cisplatin+fluorouracil; Cisplatin+topotecan; Docetaxel+carboplatin; Docetaxel+cisplatin; Doxorubicin+ifosfamide; Etoposide+cisplatin; Gemcitabine+capecitabine; Gemcitabine+cisplatin; Ifosfamide, carboplatin, etoposide; Irinotecan, fluorouracil, folinic acid; Oxaliplatin, fluorouracil, folinic acid; Paclitaxel+carboplatin; Pemetrexed+carboplatin; Pemetrexed+cisplatin; Docetaxel+cyclophosphosphamide; Vinorelbine+carboplatin; and Vinorelbine+cisplatin.

Statistical Analysis

All data were expressed as means±standard deviations. Statistical analyses were performed with GraphPad Prism 7 (San Diego, Calif.). Data were analyzed by ANOVA test followed by recommended post-hoc multiple comparisons tests. A p value of <0.05 was considered statistically significant.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

It is intended that that the scope of the present methods and compositions be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

1. A method to prepare a drug-loaded liposome comprising the steps of:
a. preparing a solution of mixed lipids in an organic medium comprising chloroform and methanol;
b. evaporating said organic medium and forming a film of the mixed lipids;
c. hydrating said film of the mixed lipids with an aqueous medium;
d. sonicating and then extruding hydrated film of the mixed lipids through a membrane followed by centrifugation to afford a liposome pellet; and
e. loading a drug by incubating said liposome pellet in a drug solution for a period of time at an elevated temperature to afford a drug-loaded liposome.

2. The method to prepare a drug-loaded liposome according to claim 1, wherein said aqueous medium comprises sodium chloride at a concentration of about 400 to 600 mM.

3. The method to prepare a drug-loaded liposome according to claim 1, wherein said mixed lipids comprises of DPPC, cholesterol and DSPE-PEG2000.

4. The method to prepare a drug-loaded liposome according to claim 1, wherein said aqueous medium comprises ammonium sulfate at a concentration of about 200 to 300 mM and sodium chloride at a concentration of about 400 to 600 mM.

5. The method to prepare a drug-loaded liposome according to claim 1, wherein said drug solution comprises gemcitabine, doxorubicin, or a combination thereof.

6. The method to prepare a drug-loaded liposome according to claim 5, wherein said drug solution comprises about 10 mg/mL of gemcitabine or doxorubicin.

7. The method to prepare a drug-loaded liposome according to claim 5, wherein said drug solution comprises about 10 mg/mL of gemcitabine and about 10 mg/mL of doxorubicin.

8. The method to prepare a drug-loaded liposome according to claim 5, wherein said drug solution comprises about 50 mg/mL of gemcitabine or doxorubicin in deionized water.

9. (canceled)

10. The method to prepare a drug-loaded liposome according to claim 1, wherein said drug solution comprises gemcitabine, platinum compounds (carboplatin, cisplatin and oxaplatin), anthracyclines (doxorubicin and daunorubicin), paclitaxel, docetaxel, camptothecin derivatives, antimetabolites (methotrexate, cytarabine), Vinca alkaloids (vincristine, vinblastine and vinorelbine), or a combination thereof.

11.-18. (canceled)

19. The method to prepare a drug-loaded liposome according to claim 10, wherein said drug solution comprises gemcitabine, capecitabine, or a combination thereof.

20. The method to prepare a drug-loaded liposome according to claim 10, wherein said drug solution comprises gemcitabine, cisplatin, or a combination thereof.

21.-31. (canceled)

32. A pharmaceutical composition comprising drug-loaded liposomes manufactured according to the following steps, together with one or more diluents, excipients or carriers:
a. preparing a solution of mixed lipids in an organic medium comprising chloroform and methanol;
b. evaporating said organic medium and forming a film of the mixed lipids;
c. hydrating said film of the mixed lipids with an aqueous medium;
d. sonicating and then extruding hydrated film of the mixed lipids through a membrane followed by centrifugation to afford a liposome pellet; and
e. loading a drug by incubating said liposome pellet in a drug solution for a period of time at an elevated temperature to afford a drug-loaded liposome.

33. The pharmaceutical composition according to claim 32, wherein said pharmaceutical composition is for treating a patient with cancer.

34. A drug-loaded liposome manufactured according to the steps of:
a. preparing a solution of mixed lipids in an organic medium of chloroform and methanol;
b. evaporating said organic medium and forming a film of the mixed lipids comprising DPPC, cholesterol and DSPE-PEG2000 at a weight ratio of about 6:3:1;
c. hydrating said film of the mixed lipids with an aqueous medium;
d. sonicating and then extruding hydrated film of the mixed lipids through a membrane followed by centrifugation to afford liposome pellet; and
e. incubating said liposome pellet in a drug solution for a period of time at an elevated temperature and affording a drug-loaded liposome.

35. The drug-loaded liposome according to claim 34, wherein said aqueous medium comprises ammonium sulfate at a concentration of about 200 to 300 mM.

36. The drug-loaded liposome according to claim 34, wherein said aqueous medium comprises sodium chloride at a concentration of about 400 to 600 mM.

37. The drug-loaded liposome according to claim 34, wherein said DPPC, cholesterol and DSPE-PEG2000 have a weight ratio of about 6:3:1.

38. The drug-loaded liposome according to claim 34, wherein said drug solution comprises a drug selected from the group consisting of gemcitabine; platinum compounds (carboplatin, cisplatin and oxaplatin), anthracyclines (doxorubicin and daunorubicin), paclitaxel, docetaxel, camptothecin derivatives, antimetabolites (methotrexate, cytarabine), and Vinca alkaloids (vincristine, vinblastine and vinorelbine).

39. (canceled)

40. (canceled)

41. The drug-loaded liposome according to claim 34, wherein said drug solution comprises gemcitabine or doxorubicin, or a combination thereof.

42.-45. (canceled)

46. The pharmaceutical composition according to claim 32, wherein said drug solution comprises gemcitabine, doxorubicin, or a combination thereof.
